(19) United States
(12) Patent Application Publication
(10) Pub. No.: US 2007/0281327 A1
(43) Pub. Date: Dec. 6, 2007

(54) Methods of Screening for Modified Antibodies With Agonistic Activities

(76) Inventors: Kiyotaka Nakano, Ibaraki (JP); Junichi Nezu, Ibaraki (JP); Takeshi Yoshino, Ibaraki (JP); Mikiyoshi Saito, Shizuoka (JP); Tetsuro Orita, Ibaraki (JP)

Correspondence Address: FISH & RICHARDSON PC, P.O. BOX 1022, MINNEAPOLIS, MN, 55440-1022, US

(21) Appl. No.: 10/582,176

(22) PCT Filed: Dec. 10, 2004
(86) PCT No.: PCT/JP04/18499

§ 371 Date: Apr. 18, 2007

(30) Foreign Application Priority Data
Dec. 12, 2003 (JP) 2003-415733

(51) Int. Cl. C07K 16/28; C12P 21/00; C12Q 1/02; G01N 33/53

(52) U.S. Cl. 435/7.92; 435/29; 435/69.1; 530/387.1

(57) ABSTRACT

Anti-human Mpl antibodies were prepared, and from these three types of antibodies with strong binding activity were selected. An expression system for single-chain antibodies derived from these selected antibodies was constructed using genetic engineering techniques. The anti-human Mpl antibodies and anti-human Mpl single-chain antibodies were assessed for TPO-like agonist activity using BaF3-human Mpl that proliferates TPO-dependently. It was found that while the anti-human Mpl antibodies did not exhibit agonistic activity, the anti-human Mpl single-chain antibodies showed agonistic activity. This shows that when screening for modified antibodies with agonistic activity, it is beneficial to determine agonistic activity after modifying antibodies with antigen-binding activity.

### CROSS-REFERENCE TO RELATED APPLICATIONS

This application is the National Stage of International Application No. PCT/JP2004/018499, filed on Dec. 10, 2004, which claims the benefit of Japanese Patent Application Serial No. 2003-415733, filed on Dec. 12, 2003. The contents of both of the foregoing applications are hereby incorporated by reference in their entireties.

### TECHNICAL FIELD

The present invention relates to methods of screening for modified antibodies with agonistic activities.

### BACKGROUND ART

Antibodies receive attention as pharmaceuticals due to their high stability and low antigenicity in blood. Of these, agonist antibodies which are capable of recognizing cell surface-expressed proteins such as receptors, and thereby induce specific reactions in cells are considered to be useful as pharmaceuticals. Several agonist antibodies such as agonist antibodies against erythropoietin receptor (see Non-patent Document 1), thrombopoietin receptor or CD47 (see Patent Documents 1 and 2) have been reported.

Recent years have seen modified antibodies with some sort of alteration such as a substitution in amino acid sequence, developed as pharmaceuticals with advantages in terms of antigenicity in humans, half-life in blood, convenience of production, and the like. For example, modified antibodies such as minibodies, humanized antibodies and chimerized antibodies are expected to have superior characteristics as pharmaceuticals. As described above, modified antibodies with agonistic activity are expected to be very useful in diagnosing and treating diseases, and thus it is necessary to develop efficient methods of screening for such antibodies.

In general, modified antibodies with agonistic activity have conventionally been screened by:
(1) preparing antibodies;
(2) determining the binding activity and agonistic activity of the prepared antibodies;
(3) selecting antibodies with binding activity and agonistic activity;
(4) modifying the antibodies;
(5) determining the binding activity and agonistic activity of the modified antibodies; and
(6) selecting those antibodies with binding activity and agonistic activity.
   This screening procedure excludes antibodies with little or no agonistic activity at a stage prior to modification, and thus the antibodies are unmodified.

Therefore, conventional screening methods do not allow discovery of antibodies that potentially have agonistic activity, but that do not have agonistic activity prior to modification.

[Patent Document 1] WO 02/33072

[Patent Document 2] WO 02/33073

[Non-patent Document 1] Elliott S et al., J. Biol. Chem., 1996, Vol. 271(40), p. 24691-24697

### DISCLOSURE OF THE INVENTION

The present invention was achieved in view of the above background. An objective of the present invention is to provide methods of screening for modified antibodies with agonistic activities. More specifically, an objective of the present invention is to provide screening methods that comprise determining agonistic activity after modifying antibodies screened using antigen-binding activity as an indicator.

The present inventors conducted dedicated studies to achieve the objectives described above. Specifically, the inventors prepared anti-human Mpl antibodies and selected antibodies with strong binding activity. The inventors then used genetic engineering techniques to construct an expression system for single-chain antibodies derived from these antibodies. Whole anti-human Mpl antibodies and anti-human Mpl single-chain minibodies were examined for TPO-like agonist activity using BaF3-human Mpl, which proliferates in a TPO-dependent manner. The anti-human Mpl single-chain minibodies exhibited agonistic activity, while the whole anti-human Mpl antibodies showed no agonistic activity.

The present inventors noted that agonistic activity differed before and after antibody modification, and that minibodies or such modified antibodies may exhibit agonistic activity when converted from antibodies that did not have agonistic activity prior to antibody modification. The inventors found that when screening for modified antibodies with agonistic activity, antibodies that could not be selected by conventional screening methods could be selected by determining agonistic activity after modifying antibodies with antigen-binding activity.

Specifically, it is impossible to discover antibodies that do not have agonistic activity prior to antibody modification, but that do have agonistic activity after modification, by using conventional screening methods in which agonistic activity is determined prior to antibody modification and antibodies that do not have agonistic activity are excluded at this point. In contrast, antibodies that would be missed by conventional methods can be discovered using screening methods which do not exclude any antibodies using agonistic activity as an indicator prior to antibody modification.

Specifically, the present invention relates to methods of screening for agonist antibodies, more specifically to:

[1] a method of screening for an agonist antibody, which comprises the steps of:

(a) determining the binding activity of a test antibody and selecting an antibody with binding activity,

(b) modifying the antibody selected in step (a), and

(c) determining the agonistic activity of the modified antibody of step (b) and selecting an antibody with agonistic activity;

[2] the screening method of [1], wherein the modified antibody is a minibody;

[3] the screening method of [2], wherein the minibody is an sc(Fv)2;

[4] the screening method of any one of [1] to [3], wherein the agonistic activity is not determined prior to modifying the test antibody;

[5] the screening method of any one of [1] to [4], wherein the antibody is one against a protein expressed on a cell membrane;

[6] an antibody obtained by the method of any one of [1] to [5];

[7] a method for producing an antibody with agonistic activity, which comprises the steps of:

(a) determining the binding activity of an antibody and selecting an antibody with binding activity,

(b) modifying the antibody selected in step (a),

(c) determining the agonistic activity of the modified antibody of step (b) and selecting an antibody with agonistic activity,

(d) introducing a host cell with a vector carrying a DNA that encodes the antibody selected in step (c), and

(e) culturing the host cell of step (d);

[8] the production method of [7], wherein the modified antibody is a minibody;

[9] the production method of [8], wherein the minibody is an sc(Fv)2;

[10] the production method of any one of [7] to [9], wherein the agonistic activity is not determined prior to antibody modification;

[11] the production method of any one of [7] to [10], wherein the antibody is one against a protein expressed on a cell membrane;

[12] a method of screening for an agonist antibody, wherein the agonistic activity of a test antibody is not determined prior to step (a), and which comprises the steps of:

(a) modifying a test antibody, and

(b) determining the agonistic activity of the modified antibody of step (a) and selecting an antibody with agonistic activity;

[13] the screening method of [12], wherein the modified antibody is a minibody; and

[14] the screening method of [13], wherein the minibody is an sc(Fv)2.

### BRIEF DESCRIPTION OF THE DRAWINGS

FIG. 1 shows the process of preparing a single-chain antibody sc(Fv)2.

FIG. 2 shows the results of evaluating VA130 sc(Fv)2 for binding activity using an Mpl-expressing CHO cell line. A purified sample of VA130 sc(Fv)2 was used.

FIG. 3 shows the results of evaluating VA130 antibody for agonistic activity using BaF3-human Mpl.

### DETAILED DESCRIPTION

The present invention provides methods of screening for modified antibodies with agonistic activity. The screening methods of the present invention comprise screening antibodies using antigen-binding activity as an indicator, modifying the screened antibodies, determining the agonistic activity of the modified antibodies, and selecting those modified antibodies with agonistic activity.

In the methods of the present invention, first, the antigen-binding activities of test antibodies are determined to select those antibodies with antigen-binding activity. The selected antibodies are then modified. Subsequently, the agonistic activities of the modified antibodies are determined to select those modified antibodies with agonistic activity.

Herein, antibody modification refers to altering an antibody's amino acid sequence, molecular weight, three-dimensional structure, and such. Antibody modification also includes, for example, low-molecular-weight conversion, chimerization/humanization, modification, and sugar chain substitution, addition, and deletion. An antibody modification may comprise multiple modifications or a single modification.

In the present invention, a preferred antibody modification is a conversion to a low-molecular-weight antibody. In preferred embodiments, such a low-molecular-weight conversion is a conversion to a diabody or sc(Fv)2. A particularly preferred low-molecular-weight conversion is a conversion to sc(Fv)2.

There is no limit as to the type of minibody, as long as it has antigen-binding activity and comprises an antibody fragment that lacks a portion of a whole antibody (for example, whole IgG or such). The antibody fragments of the present invention are not particularly limited, as long as they are portions of whole antibodies. A preferred fragment comprises a heavy chain variable region (VH) and/or a light chain variable region (VL). The amino acid sequence of a VH or VL may comprise substitutions, deletions, additions, and/or insertions. Furthermore, it is possible to delete a portion(s) of a VH and/or VL, as long as it still has antigen-binding activity. Alternatively, the variable regions may be chimerized or humanized.

The antibody fragments include Fab, Fab′, F(ab′)2, and Fv.

The minibodies include Fab, Fab′, F(ab′)2, Fv, scFv (single-chain Fv), diabody, and sc(Fv)2. Preferred minibodies are diabodies and sc(Fv)2, and particularly preferred minibodies are sc(Fv)2. Such minibodies can be produced by methods known to those skilled in the art.

The diabodies are dimers consisting of two fragments (for example, scFv) comprising variable regions linked via a linker, and typically comprise two VLs and two VHs (P. Holliger et al., Proc. Natl. Acad. Sci. USA, 90, 6444-6448 (1993); EP 404097; WO 93/11161; Johnson et al., Method in Enzymology, 203, 88-98, (1991); Holliger et al., Protein Engineering, 9, 299-305, (1996); Perisic et al., Structure, 2, 1217-1226, (1994); John et al., Protein Engineering, 12(7), 597-604, (1999); Holliger et al,. Proc. Natl. Acad. Sci. USA., 90, 6444-6448, (1993); Atwell et al., Mol. Immunol. 33, 1301-1312, (1996)).

sc(Fv)2 are antibodies consisting of single-chain polypeptides in which two heavy chain variable regions are linked with two light chain variable regions via linkers or the like (Hudson et al, J Immunol. Methods 1999; 231:177-189). sc(Fv)2 can be prepared, for example, by linking two sc(Fv) via a linker.

The order of the two heavy chain variable regions and two light chain variable regions which are linked together is not particularly limited, and the regions may be arranged in any order. Examples of arrangements are listed below:
[VL] linker [VH] linker [VH] linker [VL]
[VH] linker [VL] linker [VL] linker [VH]
[VH] linker [VH] linker [VL] linker [VL]
[VH] linker [VL] linker [VH] linker [VL]
[VL] linker [VL] linker [VH] linker [VH]
[VL] linker [VH] linker [VL] linker [VH]

In the present invention, a preferred sc(Fv)2 is arranged: [VH] linker [VL] linker [VH] linker [VL].

The linkers comprise arbitrary peptide linkers that can be introduced using genetic engineering or synthetic linkers (for example, linkers disclosed in “Protein Engineering, 9(3), 299-305, 1996”).

The preferred linkers in the present invention are peptide linkers. The lengths of the peptide linkers are not particularly limited and those skilled in the art can appropriately select the lengths depending on the purpose. Typical lengths are one to 100 amino acids, preferably 5 to 30 amino acids, and particularly preferably 12 to 18 amino acids (for example, 15 amino acids).

Amino acid sequences of such peptide linkers include, for example:
Ser
Gly•Ser
Gly•Gly•Ser
Ser•Gly•Gly
Gly•Gly•Gly•Ser
Ser•Gly•Gly•Gly
Gly•Gly•Gly•Gly•Ser
Ser•Gly•Gly•Gly•Gly
Gly•Gly•Gly•Gly•Gly•Ser
Ser•Gly•Gly•Gly•Gly•Gly
Gly•Gly•Gly•Gly•Gly•Gly•Ser
Ser•Gly•Gly•Gly•Gly•Gly•Gly
(Gly•Gly•Gly•Gly•Ser)n
(Ser•Gly•Gly•Gly•Gly)n
   where n is an integer of 1 or larger.

Synthetic linkers (chemical crosslinking agents) include crosslinking agents that are routinely used to crosslink peptides, for example, N-hydroxy succinimide (NHS), disuccinimidyl suberate(DSS), bis(succinimidyl) suberate(BS³), dithiobis(succinimidyl propionate)(DSP), dithiobis(succinimidyl propionate)(DTSSP), ethylene glycol bis(succinimidyl succinate)(EGS), ethylene glycol bis(sulfosuccinimidyl succinate)(sulfo-EGS), disuccinimidyl tartrate(DST), disulfosuccinimidyl tartrate(sulfo-DST), bis[2-(succinimidoxycarbonyloxy)ethyl] sulfone(BSOCOES), and bis[2-(succinimidoxycarbonyloxy)ethyl] sulfone(sulfo-BSOCOES). These crosslinking agents are commercially available.

The antibody fragments or minibodies can be obtained by treating antibodies with an enzyme, for example, papain or pepsin. Alternatively, genes encoding the antibody fragments may be constructed and inserted into expression vectors, and then expressed in appropriate host cells (see, for example, Co, M. S. et al., J. Immunol. (1994) 152, 2968-2976; Better, M. and Horwitz, A. H., Methods Enzymol. (1989) 178, 476-496; Pluckthun, A. and Skerra, A., Methods Enzymol. (1989) 178, 497-515; Lamoyi, E., Methods Enzymol. (1986) 121, 652-663; Rousseaux, J. et al., Methods Enzymol. (1986) 121, 663-669; Bird, R. E. and Walker, B. W., Trends Biotechnol. (1991) 9, 132-137).

The molecular weight of a minibody of the present invention is preferably less than that of a whole antibody. However, the minibodys may exist, for example, as multimers such as dimers, trimers, or tetramers, and therefore their molecular weight may be greater than that of a whole antibody.

The chimeric antibodies are antibodies prepared by combining sequences derived from different animals, which include, for example, antibodies comprising the heavy and light chain variable regions of a mouse antibody and the heavy and light chain constant regions of a human antibody. The chimeric antibodies can be prepared by known methods. To obtain such chimeric antibodies, a DNA encoding an antibody V region can be ligated with a DNA encoding a human antibody constant region; the resulting ligation product can be inserted into an expression vector; and the construct can be introduced into a host to produce the antibody.

Humanized antibodies are also referred to as reshaped human antibodies, and obtained by substituting the complementarity determining region (CDR) of a human antibody for the complementarity determining region of an antibody derived from a nonhuman mammal, for example, a mouse. General gene recombination techniques are also known for this (see European Patent Application No. 125023; and WO 96/02576).

Specifically, a DNA sequence is designed to comprise a mouse antibody CDR and a human antibody framework region (FR) linked together, and then synthesized by PCR using as primers several oligonucleotides prepared to comprise portions that overlap the edges of both the CDR and FR (see the method described in WO 98/13388).

Human antibody framework regions linked via the CDR are selected so that the complementarity determining region forms a suitable antigen-binding domain. Amino acids in the framework region of the antibody variable region can be substituted as required so that the complementarity determining region of the reshaped human antibody forms a suitable antigen-binding domain (Sato, K. et al., Cancer Res. (1993) 53, 851-856).

The constant regions of human antibodies can be used as the constant regions of chimeric antibodies and humanized antibodies, and include, for example, Cγ1, Cγ2, Cγ3, and Cγ4 for the H chain, and Cκ and Cλ, for the L chain. Alternatively, human antibody constant regions may be modified to improve an antibody or the stability of its production.

In general, chimeric antibodies comprises a variable region of an antibody derived from a nonhuman mammal, and a constant region of a human antibody. Humanized antibodies generally comprise the complementarity determining region of an antibody derived from a nonhuman mammal, and the framework region and constant region of a human antibody.

Amino acids in the variable region (for example, FR) and/or constant region can be substituted with other amino acids after preparing a chimeric antibody or humanized antibody.

An antibody can be modified and altered by adding other molecules to the antibody. Such antibody modifications can be achieved by methods known to those skilled in the art and include, for example, the addition of macromolecules such as PEG.

An antibody can also be modified by substituting, adding, or removing sugar chains. Techniques for sugar chain modification are already known to those skilled in the art (for example, WO 00/61739 and WO 02/31140).

The test antibodies for the screening methods of the present invention are not particularly limited and may be any type of antibody.

The test antibodies are not limited by their origin or such. The antibodies include those derived from any animal, for example, mouse antibodies, human antibodies, rat antibodies, rabbit antibodies, camel antibodies.

The test antibodies in the present invention are preferably unmodified antibodies (for example, whole antibodies). However, modified antibodies may be used as test antibodies. When using a modified antibody as a test antibody, additional modifications are carried out during the screening process of the present invention. In such cases, the same or different types of modification can be carried out.

Thus, the test antibodies include, for example, antibodies whose amino acid sequences comprise substitutions, such as chimeric antibodies and humanized antibodies; antibody modification products linked with various molecules; antibodies whose glycosylation state has been altered; and minibodies. However, when a test antibody is a minibody, the minibody resulting from the modification is preferably a diabody or sc(Fv)2. Thus, the test antibodies prior to modification are preferably antibodies other than diabodies and sc(Fv)2.

The antigens recognized by the antibodies of the present invention are not particularly limited, and the antibodies may recognize any type of antigen, including, for example, proteins expressed on cell membranes or in cells. Such proteins expressed on cell membranes or in cells include, for example, receptors, cell surface antigens, and major histocompatibility antigens.

The receptors include, for example, receptors that belong to receptor families such as the hematopoietic factor receptor family, cytokine receptor family, tyrosine kinase receptor family, serine/threonine kinase receptor family, TNF receptor family, G protein-coupled receptor family, GPI-anchored receptor family, tyrosine phosphatase receptor family, adhesion factor family, and hormone receptor family. Various references that relate to receptors belonging to these receptor families and their characteristics are available and include, for example, Cooke B A., King R J B., van der Molen H J. ed. New Comprehensive Biochemistry Vol. 18B “Hormones and their Actions Part II” pp. 1-46 (1988) Elsevier Science Publishers BV., New York, USA; Patthy L. (1990) Cell, 61: 13-14; Ullrich A., et al. (1990) Cell, 61: 203-212; Massagul J. (1992) Cell, 69: 1067-1070; Miyajima A., et al. (1992)Annu. Rev. Immunol., 10: 295-331; Taga T. and Kishimoto T. (1992) FASEB J., 7: 3387-3396; Fantl W I., et al. (1993) Annu. Rev. Biochem., 62: 453-481; Smith C A., et al. (1994) Cell, 76: 959-962; Flower D R. (1999) Biochim. Biophys. Acta, 1422: 207-234; and M. Miyasaka ed., Cell Technology, supplementary volume, Handbook series, “Handbook for Adhesion Factors” (1994) (Shujunsha, Tokyo, Japan).

Specifically, receptors belonging to the above-described receptor families include, for example, the following human and mouse receptors: erythropoietin (EPO) receptors, granulocyte colony-stimulating factor (G-CSF) receptors, thrombopoietin (TPO) receptors, insulin receptors, Flt-3 ligand receptors, platelet-derived growth factor (PDGF) receptors, interferon (IFN)-α and -β receptors, leptin receptors, growth hormone (GH) receptors, interleukin (IL)-10 receptors, insulin-like growth factor (IGF)-I receptors, leukemia inhibitory factor (LIF) receptors, and ciliary neurotrophic factor (CNTF) receptors (hEPOR: Simon, S. et al. (1990) Blood 76, 31-35.; mEPOR: D'Andrea, A D. et al. (1989) Cell 57, 277-285; hG-CSFR: Fukunaga, R. et al. (1990) Proc. Natl. Acad. Sci. USA. 87, 8702-8706; mG-CSFR: Fukunaga, R. et al. (1990) Cell 61, 341-350; hTPOR: Vigon, I. et al. (1992) 89, 5640-5644; mTPOR: Skoda, R C. et al. (1993) 12, 2645-2653; hInsR: Ullrich, A. et al. (1985) Nature 313, 756-761; hFlt-3: Small, D. et al. (1994) Proc. Natl. Acad. Sci. USA. 91, 459-463; hPDGFR: Gronwald, R G K. et al. (1988) Proc. Natl. Acad. Sci. USA. 85, 3435-3439; hIFN α/βR: Uze, G. et al. (1990) Cell 60, 225-234; and Novick, D. et al. (1994) Cell 77, 391-400).

The major histocompatibility antigens include, for example, MHC class I (HLA-A, HLA-B, HLA-C, HLA-E, HLA-F, HLA-G, and HLA-H) and class II antigens (HLA-DR, -DQ, and -DP).

The cell surface antigens include, for example, CD1, CD2, CD3, CD4, CD5, CD6, CD7, CD8, CD10, CD11a, CD11b, CD11c, CD13, CD14, CD15s, CD16, CD18, CD19, CD20, CD21, CD23, CD25, CD28, CD29, CD30, CD32, CD33, CD34, CD35, CD38, CD40, CD41a, CD41b, CD42a, CD42b, CD43, CD44, CD45, CD45RO, CD48, CD49a, CD49b, CD49c, CD49d, CD49e, CD49f, CD51, CD54, CD55, CD56, CD57, CD58, CD61, CD62E, CD62L, CD62P, CD64, CD69, CD71, CD73, CD95, CD102, CD106, CD122, CD126, and CDw130.

In the screening methods of the present invention, for example, when the number of test antibodies is small, the antibodies can be modified before determining their binding activities, then their agonistic activities and binding activities can be determined, and then modified antibodies with agonistic activity can be selected. In this case, the order in which agonistic activity and binding activity are determined is not particularly limited, and it is possible to determine only the agonistic activity.

In one preferred embodiment, the screening methods of the present invention are screening methods comprising the use of a whole antibody as a test antibody that has not been subjected to low-molecular-weight conversion, and at the modification step, it is converted to a low-molecular-weight antibody (for example, a diabody or sc(Fv)2).

Herein, agonistic activity refers to an activity caused by antibody binding that induces a specific reaction in cells (for example, inducing a change in a certain physiological activity by transmitting a signal into a cell). The physiological activities include, for example, growth activities, growth-inducing activities, survival activities, differentiation activities, differentiation-inducing activities, transcriptional activities, membrane transport activities, binding activities, proteolytic activities, phosphorylation/dephosphorylation activities, oxidation-reduction activities, transfer activities, nucleolytic activities, dehydration activities, cell death-inducing activities, and apoptosis-inducing activities, but are not limited thereto.

The agonistic activity can be determined by methods known to those skilled in the art.

For example, agonistic activity can be evaluated by methods which use cell growth as an indicator, as described in the Examples. More specifically, an antibody whose agonistic activity is to be determined is added to cells that show agonist-dependent growth, and the cells are cultured. Next, a reagent that shows a color reaction at a particular wavelength depending on viable cell count, such as WST-8, is added, and the absorbance is measured. The agonistic activity can be determined using the measured absorbance as an indicator.

The cells showing agonist-dependent growth can also be prepared by methods known to those skilled in the art. For example, when the antigen is a receptor that transduces a cell growth signal, cells that express that receptor can be used. Alternatively, when the antigen is a receptor that does not transduce a cell growth signal, it is possible to prepare a chimeric receptor comprising the intracellular domain of a receptor that transduces a cell growth signal and the extracellular domain of a receptor that does not, and to express the resulting chimeric receptors in cells. Such receptors that transduce cell growth signals include, for example, G-CSF receptor, mpl, neu, GM-CSF receptor, EPO receptor, c-kit, and FLT-3. The cells used to express the receptors include, for example, BaF3, NFS60, FDCP-1, FDCP-2, CTLL-2, DA-1, and KT-3.

In addition, there is no limitation as to the type of detection indicators to be used for determining agonistic activity, as long as the indicator can monitor quantitative and/or qualitative changes. For example, it is possible to use cell-free assay indicators, cell-based assay indicators, tissue-based assay indicators, and in vivo assay indicators. Indicators that can be used in cell-free assays include enzymatic reactions, quantitative and/or qualitative changes in proteins, DNAs, or RNAs. Such enzymatic reactions include, for example, amino acid transfers, sugar transfers, dehydrations, dehydrogenations, and substrate cleavages. Alternatively, protein phosphorylations, dephosphorylations, dimerizations, multimerizations, hydrolyses, dissociations and such; DNA or RNA amplifications, cleavages, and extensions can be used as the indicator in cell-free assays. For example, protein phosphorylations downstream of a signal transduction pathway may be used as a detection indicator. Alterations in cell phenotype, for example, quantitative and/or qualitative alterations in products, alterations in growth activity, alterations in cell number, morphological alterations, or alterations in cellular properties, can be used as the indicator in cell-based assays. The products include, for example, secretory proteins, surface antigens, intracellular proteins, and mRNAs. The morphological alterations include, for example, alterations in dendrite formation and/or dendrite number, alteration in cell flatness, alteration in cell elongation/axial ratio, alterations in cell size, alterations in intracellular structure, heterogeneity/homogeneity of cell populations, and alterations in cell density. Such morphological alterations can be observed under a microscope. Cellular properties to be used as the indicator include anchor dependency, cytokine-dependent response, hormone dependency, drug resistance, cell motility, cell migration activity, pulsatory activity, and alteration in intracellular substances. Cell motility includes cell infiltration activity and cell migration activity. The alterations in intracellular substances include, for example, alterations in enzyme activity, mRNA levels, levels of intracellular signaling molecules such as Ca²⁺ and cAMP, and intracellular protein levels. When a cell membrane receptor is used, alterations in the cell proliferating activity induced by receptor stimulation can be used as the indicator. The indicators to be used in tissue-based assays include functional alterations adequate for the subject tissue. In in vivo assays, alterations in tissue weight, alterations in the blood system (for example, alterations in blood cell counts, protein contents, or enzyme activities), alterations in electrolyte levels, and alterations in the circulating system (for example, alterations in blood pressure or heart rate).

The methods for measuring such detection indicators are not particularly limited. For example, absorbance, luminescence, color development, fluorescence, radioactivity, fluorescence polarization, surface plasmon resonance signal, time-resolved fluorescence, mass, absorption spectrum, light scattering, and fluorescence resonance energy transfer may be used. These measurement methods are known to those skilled in the art and may be selected appropriately depending on the purpose. For example, absorption spectra can be obtained by using a conventional photometer, plate reader, or such; luminescence can be measured with a luminometer or such; and fluorescence can be measured with a fluorometer or such. Mass can be determined with a mass spectrometer. Radioactivity can be determined with a device such as a gamma counter depending on the type of radiation. Fluorescence polarization can be measured with BEACON (TaKaRa). Surface plasmon resonance signals can be obtained with BIACORE. Time-resolved fluorescence, fluorescence resonance energy transfer, or such can be measured with ARVO or such. Furthermore, a flow cytometer can also be used for measuring. It is possible to use one of the above methods to measure two or more different types of detection indicators. A greater number of detection indicators may also be examined by using two or more measurement methods simultaneously and/or consecutively. For example, fluorescence and fluorescence resonance energy transfer can be measured at the same time with a fluorometer.

The binding activity of an antibody can be determined by methods known to those skilled in the art. Methods for determining the antigen-binding activity of an antibody include, for example, enzyme-linked immunosorbent assays (ELISAs), enzyme immunoassays (EIAs), radioimmunoassays (RIAs), and fluorescent antibody methods. For example, when an enzyme immunoassay is used, samples comprising an antibody, for example, a culture supernatant of antibody-producing cells or a purified antibody, are added to plates coated with an antigen. A secondary antibody labeled with an enzyme such as alkaline phosphatase is added, and the plates are incubated. After washing, an enzyme substrate such as p-nitrophenyl phosphate is added and the resulting absorbance is determined to evaluate the antigen-binding activity.

The present invention also provides methods for producing antibodies with agonistic activity. In the production methods of the present invention, modified antibodies with agonistic activity are first screened as described above. Next, vectors carrying DNA encoding the modified antibodies are prepared and introduced into host cells. Then, the host cells are cultured.

For example, when E. coli is used as a host, the vectors of the present invention are not particularly limited, as long as they have an “ori” for amplification in E. coli (for example, JM109, DH5α, HBO101, and XL1Blue) or such, which allows large-scale preparation, and a gene for selecting transformed E. coli (for example, a drug resistance gene that allows evaluation using an agent (ampicillin, tetracycline, kanamycin, and chloramphenicol)). The vectors include M13 vectors, pUC vectors, pBR322, pBluescript, and pCR-Script. Alternatively, when aiming to subclone and excise cDNA, the vectors include, for example, pGEM-T, pDIRECT, and pT7, in addition to the vectors described above.

Expression vectors are particularly useful as vectors of the present invention. For example, when aiming for expression in E. coli such as JM109, DH5α, HB101, and XL1-Blue, the expression vectors not only have the above-described characteristics that allow vector amplification in E. coli, but must also carry a promotor that allows efficient expression in E. coli, for example, lacZ promotor (Ward et al., Nature (1989) 341, 544-546; FASEB J. (1992) 6, 2422-2427), araB promotor (Better et al., Science (1988) 240, 1041-1043), T7 promotor or such. Such vectors include pGEX-5X- 1 (Pharmacia), “QIAexpress system” (Quiagen), pEGFP, or pET (in this case, the host is preferably BL21 that expresses T7 RNA polymerase) in addition to the vectors described above.

The vectors may comprise signal sequences for polypeptide secretion. As a signal sequence for protein secretion, a pelB signal sequence (Lei, S. P. et al J. Bacteriol. (1987) 169, 4379) may be used when a protein is secreted into the E. coli periplasm. The vector can be introduced into host cells by calcium chloride or electroporation methods, for example.

In addition to vectors for E. coli, the vectors of the present invention include mammalian expression vectors (for example, pcDNA3 (Invitrogen), pEGF-BOS (Nucleic Acids. Res.1990, 18(17), p5322), pEF, and pCDM8), insect cell-derived expression vectors (for example, the “Bac-to-BAC baculovirus expression system” (Gibco-BRL) and pBacPAK8), plant-derived expression vectors (for example, pMH1 and pMH2), animal virus-derived expression vectors (for example, pHSV, pMV, and pAdexLcw), retroviral expression vectors (for example, pZIPneo), yeast expression vectors (for example, “Pichia Expression Kit” (Invitrogen), pNV11, and SP-Q01), and Bacillus subtilis expression vectors (for example, pPL608 and pKTH50), for example.

When aiming for expression in animal cells such as CHO, COS, and NIH3T3 cells, the vectors must have a promotor essential for expression in cells, for example, SV40 promotor (Mulligan et al., Nature (1979) 277, 108), MMTV-LTR promotor, EF1α promotor (Mizushima et al., Nucleic Acids Res. (1990) 18, 5322), and CMV promotor, and more preferably they have a gene for selecting transformed cells (for example, a drug resistance gene that allows evaluation using an agent (neomycin, G418, or such). Vectors with such characteristics include pMAM, pDR2, pBK-RSV, pBK-CMV, pOPRSV, and pOP13, for example.

In addition, the following method can be used for stable gene expression and gene amplification in cells: CHO cells deficient in a nucleic acid synthesis pathway are introduced with a vector (for example, PCHOI) that carries a DHFR gene which compensates for the deficiency, and the vector is amplified using methotrexate (MTX). Alternatively, the following method can be used for transient gene expression: COS cells with a gene expressing SV40 T antigen on their chromosome are transformed with a vector (pcD and such) with an SV40 replication origin. Replication origins derived from polyoma virus, adenovirus, bovine papilloma virus (BPV), and such can also be used. To amplify gene copy number in host cells, the expression vectors may further carry selection markers such as aminoglycoside phosphotransferase (APH) gene, thymidine kinase (TK) gene, E. coli xanthine-guanine phosphoribosyltransferase (Ecogpt) gene, and dihydrofolate reductase (dhfr) gene.

In these methods, the vector is introduced into host cells as the next step. The host cells to which the vectors are introduced are not particularly limited, and for example, E. coli and various types of animal cells can be used. The host cells can be used, for example, as production systems for expressing and producing the antibodies of the present invention. The systems producing the antibodies include in vitro and in vivo production systems. The in vitro production systems include production systems using eucaryotic or procaryotic cells.

When eucaryotic cells are used, for example, animal cells, plant cells and fungal cells can be used as hosts. Such animal cells include mammalian cells (for example, CHO (J. Exp. Med. (1995) 108, 945), COS, 3T3, myeloma, baby hamster kidney (BHK), HeLa, and Vero), amphibian cells (for example, Xenopus oocyte (Valle, et al., Nature (1981) 291, 338-340)), and insect cells (for example, Sf9, Sf21, and Tn5). In the present invention, CHO-DG44, CHO-DXB11, COS7 cells, and BHK are preferably used. CHO cells are particularly preferred for large-scale expression in animal cells. The vectors can be introduced into host cells, for example, using calcium phosphate methods, DEAE-dextran methods, methods using cationic liposome DOTAP (Boehringer-Mannheim), electroporation, and lipofection.

The plant cells include, for example, Nicotiana tabacum-derived cells, which are known as protein production systems and can be callus-cultured. The fungal cells include yeasts, for example, the genus Saccharomyces such as Saccharomyces cerevisiae and Saccharomyces pombe; and filamentous bacteria, for example, the genus Aspergillus such as Aspergillus niger.

When using procaryotic cells, production systems using bacterial cells are available. Such bacterial cells include E. coli, for example, JM109, DH5α, and HB101. In addition, Bacillus subtilis can also be used.

In these methods, the above-described host cells are cultured as the next step. Antibodies are obtained by in vitro culturing of cells transformed with a DNA of interest. The cultures can be performed by known methods. For example, media that can be used for animal cells include, for example, DMEM, MEM, RPMI1640, and IMDM. Serum supplements such as FBS and fetal calf serum (FCS) may be used in the cultures. Alternatively, the cultures may be carried out using serum-free media. The pH of the cultures is preferably in the range of about 6 to 8. The cultures are typically carried out for about 15 to 200 hours at about 30° C. to 40° C., and if required, the medium is changed and aeration and stirring are provided.

In vivo polypeptide production systems include, for example, production systems using animals or plants. A DNA of interest is introduced into such an animal or plant, the animal or plant is allowed to produce the polypeptide in vivo, and the produced polypeptide is collected. Herein, the “hosts” include such animals and plants.

Such production systems using animals include those using mammals or insects. Such mammals include goats, pigs, sheep, mice, and cows (Vicki Glaser, SPECTRUM Biotechnology Applications, 1993). When using mammals, transgenic animals can also be used.

For example, a DNA of interest is prepared as a fusion gene with a gene which encodes a polypeptide inherently produced in milk, such as goat β casein. Then, goat embryos are injected with a DNA fragment comprising the fusion gene, and transplanted into female goats. The desired antibody can be obtained from milk produced by transgenic goats born of the goats that received embryos, or by their progenies. Hormones may be appropriately given to the transgenic goats to increase the amount of antibody-containing milk they produce (Ebert, K. M. et al., Bio/Technology (1994) 12, 699-702).

Silkworms are an example of the insects that can be used. By infecting a silkworm with a baculovirus that carries a DNA encoding the desired antibody, the desired antibody can be obtained from the body fluids of the silkworm (Susumu, M. et al., Nature (1985) 315, 592-594).

Tobacco is an example of the plants which can be used. When using tobacco, a DNA encoding a desired antibody is inserted into a plant expression vector, for example, pMON 530, and the vector is introduced into a bacterium such as Agrobacterium tumefaciens. The bacterium is infected to tobacco, for example, Nicotiana tabacum, and the desired antibody can be obtained from the leaves of the tobacco (Julian K.-C. Ma et al., Eur. J. Immunol. (1994) 24, 131-138).

Desired antibodies obtained by the methods described above can be isolated from inside host cells or from outside the cells (the medium, or such), and purified to homogeneity. The antibodies can be isolated and purified by methods routinely used for isolating and purifying polypeptides, and the type of method is not limited. For example, the antibodies can be isolated and purified by appropriately selecting and combining column chromatography, filtration, ultrafiltration, salting out, solvent precipitation, solvent extraction, distillation, immunoprecipitation, SDS-polyacrylamide gel electrophoresis, isoelectrofocusing, dialysis, recrystallization, and such.

The chromatography includes, for example, affinity chromatography, ion exchange chromatography, hydrophobic chromatography, gel filtration, reverse phase chromatography, and adsorption chromatography (Strategies for Protein Purification and Characterization: A Laboratory Course Manual. Ed Daniel R. Marshak et al., Cold Spring Harbor Laboratory Press, 1996). The chromatographic methods described above can be conducted using liquid chromatography, for example, HPLC and FPLC. Columns that can be used for affinity chromatography include protein A columns and protein G columns. Columns using protein A include, for example, Hyper D, POROS, and Sepharose F. F. (Pharmacia).

Appropriate modifications or partial removal of peptides can be achieved by reacting antibodies with appropriate protein modification enzymes before or after antibody purification. Such protein modification enzymes include, for example, trypsin, chymotrypsin, lysyl endopeptidase, protein kinase, and glycosidase.

The present invention also provides modified antibodies with agonistic activity obtainable by the screening methods of the present invention, and modified antibodies produced by the production methods of the present invention.

Furthermore, the screening or production methods of the present invention can be used to screen for or produce not only antibodies with agonistic activity, but also antibodies with other activities such as neutralizing activity, cytotoxic activity, binding activity, antagonistic activity, and enzymatic activity.

All prior art documents cited herein are incorporated herein by reference.

### EXAMPLES

Herein below, the present invention will be specifically described with reference to Examples.

### Example 1

### Preparation of Anti-Human Mpl Antibodies

1.1 Establishment of Mpl-Expressing BaF3 Cell Lines

BaF3 cell lines expressing the full-length Mpl gene were established to obtain cell lines that proliferate in a TPO-dependent manner. A full-length human Mpl cDNA (Palacios, R. et al., Cell, 41, 727-734 (1985)) (GenBank accession NO. NM_{—}005373) was amplified by PCR. The cDNA was cloned into a pCOS2 expression vector to construct pCOS2-hMplfull. The expression vector pCOS2 was constructed by removing the DHFR gene expression region from pCHOI (Hirata, Y et al., FEBS Letter, 356, 244-248 (1994)), where the expression region of the neomycin resistance gene HEF-VH-gγ1 (Sato, K. et al., Mol Immunol., 31, 371-381 (1994)) was inserted.

Each vector (20 μg) prepared as described above was mixed with BaF3 cells (1×10⁷ cells/mL) suspended in PBS in Gene Pulser cuvettes. This mixture was then pulsed at 0.33 kV and 950 μFD using a Gene Pulser II (Bio-Rad). The BaF3 cells introduced with the above DNAs by electroporation were added to RPMI 1640 medium (Invitrogen) containing 1 ng/mL mouse interleukin 3 (hereinafter abbreviated as mIL-3; Peprotech), 500 μg/mL Geneticin (Invitrogen), and 10% FBS (Invitrogen), and selected to establish a human Mpl-expressing BaF3 cell line (hereinafter abbreviated as “BaF3-human Mpl”). Following selection, the cells were cultured and maintained in RPMI 1640 containing 1 ng/mL rhTPO (R&D) and 10% FBS.

1.2 Establishment of Mpl-Expressing CHO Cell Lines

CHO cell lines expressing the full-length Mpl gene were established to obtain cell lines to be used for assessing binding activity by flow cytometry. First, the DHFR gene expression site from pCHOI was inserted into pCXN2 (Niwa, H. et al., Gene, 108, 193-199 (1991)) at the HindIII site to prepare a pCXND3 expression vector. The respective Mpl genes were amplified by PCR using pCOS2-hMplfull, pCOS2-monkeyMplfull, and pCOS2-mouseMplfull as templates, and primers with a His-tag sequence. The PCR products were cloned into pCXND3 to construct pCXND3-hMpl-His, and pCXND3-monkey Mpl-His, respectively.

Vectors thus prepared (25 μg each) were mixed with a PBS suspension of CHO-DG44 cells (1×10⁷ cells/mL) in Gene Pulser cuvettes. The mixture was then pulsed at 1.5 kV and 25 μFD using Gene Pulser II (Bio-Rad). The CHO cells introduced with these DNAs by electroporation were added to CHO-S-SFMII medium (Invitrogen) containing 500 μg/mL Geneticin and 1×HT (Invitrogen). A human Mpl-expressing CHO cell line (hereinafter abbreviated as “CHO-human Mpl”), and a monkey Mpl-expressing CHO cell line (hereinafter abbreviated as “CHO-monkey Mpl”) were established through selection.

1.3 Preparation of Soluble Human Mpl Protein

To prepare soluble human Mpl protein, an expression system using insect Sf9 cells for production and secretion of the protein was constructed as described below.

A DNA construct encoding the extracellular region of human Mpl (Gln 26 to Trp 491) with a downstream FLAG tag was prepared. The construct was inserted into a pBACSurf-1 Transfer Plasmid (Novagen) between the PstI and SmaI sites to prepare pBACSurf1-hMpl-FLAG. Then, Sf9 cells were transformed with 4 μg of pBACSurf1-hMpl-FLAG using the Bac-N-Blue Transfection Kit (Invitrogen). The culture supernatant was collected after a three-day incubation. Recombinant virus was isolated by plaque assays. The prepared virus stock was used to infect Sf9 cells, and the culture supernatant was collected.

Soluble human Mpl protein was purified from the obtained culture supernatant as described below. The culture supernatant was loaded onto a Q Sepharose Fast Flow (Amersham Biosciences) for adsorption, and the adsorbed protein was then eluted with 50 mM Na-phosphate buffer (pH7.2) containing 0.01% (v/v) Tween 20 and 500 mM NaCl. After the eluates were loaded onto a FLAG M2-Agarose (Sigma-Aldrich) for adsorption, the protein adsorbed was eluted with 100 mM glycine-HCl buffer (pH3.5) containing 0.01% (v/v) Tween 20. Immediately after elution, the fraction obtained was neutralized with 1 M Tris-HCl Buffer (pH8.0) and the buffer was exchanged with PBS(−) and 0.01% (v/v) Tween 20 using PD-10 columns (Amersham Biosciences). The purified soluble Mpl protein was referred to as “shMpl-FLAG”.

1.4 Preparation of Human Mpl-IgG Fc Fusion Protein

Human fusion protein Mpl-IgG Fc gene was prepared according to the method by Bennett et al. (Bennett, B. D. et al., J. Biol. Chem. 266, 23060-23067 (1991)). A nucleotide sequence encoding the extracellular region of human Mpl (Gln 26 to Trp 491) was linked to a nucleotide sequence encoding the Fc region of human IgG-γ1 (a region downstream of Asp 216). A BstEII sequence (amino acids: Val-Thr) was attached to the junction as a fusion linker between these two regions. A 19-amino acid signal peptide derived form human IgG H chain variable region was used as the signal sequence. The resulting human fusion protein Mpl-IgG Fc gene was cloned into pCXND3 to construct pCXND3-hMpl-Fc.

The vectors thus prepared (25 μg) was each mixed with a PBS suspension of CHO-DG44 cells (1×10⁷ cells/mL) in Gene Pulser cuvettes. The mixture was then pulsed at 1.5 kV and 25 μFD using Gene Pulser II (Bio-Rad). The CHO cells introduced with the DNA by electroporation were added to CHO-S-SFMII medium containing 500 μg/mL Geneticin and 1×HT (Invitrogen). shMPL-Fc-expressing CHO cell line (CHO-hMpl-Fc) was then established through selection.

Human Mpl-IgG Fc fusion protein was purified from the culture supernatant as described below. The culture supernatant was loaded onto a Q Sepharose Fast Flow (Amersham Biosciences) for adsorption, and then the adsorbed protein were eluted with 50 mM Na-phosphate buffer (pH7.6) containing 0.01% (v/v) Tween 20 and 1 M NaCl. After the eluates were loaded onto a HiTrap protein G HP column (Amersham Biosciences) for adsorption, the adsorbed protein was eluted with 0.1 M glycine-HCl buffer (pH2.7) containing 150 mM NaCl and 0.01% (v/v) Tween 20. Immediately after elution, the obtained fraction was neutralized with 1 M Tris-HCl Buffer (pH8.0) and the buffer was exchanged with PBS(−) and 0.01% (v/v) Tween 20 using PD-10 columns (Amersham Biosciences). The purified soluble Mpl protein was referred to as “hMpl-Fc”.

1.5 Immunization with shMpl-FLAG and Hybridoma Selection

MRL/MpJUmmCrj-lpr/lpr mice (hereinafter abbreviated as “MRL/lpr mice”; purchased from Charles River, Japan) were immunized; the primary immunization was carried out at eight weeks of age. For every single mouse, an emulsion containing 100 μg of shMPL-FLAG combined with Freund's complete adjuvant (H37 Ra; Beckton Dickinson), was administered subcutaneously as the primary injection. As a booster injection, an emulsion containing shMPL-FLAG (50 μg per mouse) combined with Freund's incomplete adjuvant (Beckton Dickinson) was administered subcutaneously. Three mice which have been immunized six times in total were subjected to a final injection of shMPL-FLAG (50 μg per mouse) through the caudal vein. Cell fusion was achieved by mixing the mouse myeloma P3-X63Ag8U1 cells (P3U1; purchased from ATCC) and mouse splenocytes using polyethylene glycol 1500 (Roche Diagnostics). Hybridoma selection in HAT medium began the following day and culture supernatants were obtained. Screening was carried out by ELISA, using immunoplates immobilized with shMpl-FLAG or hMpl-Fc and the assayed cell proliferation activity of BaF3-hMpl as an indicator. Positive clones were isolated as single clones by limiting dilution and then cultured on a large scale. The culture supernatants were then collected.

1.6 Analyses of Anti-Human Mpl Antibodies

Antibody concentrations were determined by carrying out a mouse IgG sandwich ELISA using goat anti-mouse IgG (gamma) (ZYMED) and alkaline phosphatase-goat anti-mouse IgG (gamma) (ZYMED), generating a calibration curve by GraphPad Prism (GraphPad Software; USA), and calculating the antibody concentrations from the calibration curve. Commercially available antibodies of the same isotype were used as standards.

Antibody isotypes were determined by antigen-dependent ELISA using isotype-specific secondary antibodies. hMpl-Fc was diluted to 1 μg/mL with a coating buffer (0.1 mM NaHCO₃, pH9.6) containing 0.02% (w/v) NaN₃, and then added to ELISA plates. The plates were incubated overnight at 4° C. for coating. The plates were blocked with a diluent buffer (50 mM Tris-HCl (pH8.1) containing 1 mM MgCl₂, 150 mM NaCl, 0.05% (v/v) Tween 20, 0.02% (w/v) NaN₃, 1% (w/v) BSA). After the addition of hybridoma culture supernatants, the plates were allowed to stand at room temperature for 1 hr. After washing with a rinse buffer (0.05% (v/v) Tween 20 in PBS), alkaline phosphatase-labeled isotype-specific secondary antibodies were added to the plates. Then, the plates were allowed to stand at room temperature for 1 hr. Color development was carried out using SIGMA 104 (Sigma-Aldrich) diluted to 1 mg/mL with a substrate buffer (50 mM NaHCO₃, pH9.8) containing 10 mM MgCl₂, and absorbance was measured at 405 nm using Benchmark Plus (Bio-Rad).

The binding activities of an antibody to shMpl-FLAG and hMPL-Fc were determined by ELISA. ELISA plates were coated with 1 μg/mL of purified shMpl-FLAG or hMPL-Fc, and blocked with a diluent buffer. Hybridoma culture supernatants were added to the plates, and the plates were allowed to stand at room temperature for I hr. Then, alkaline phosphatase-labeled anti-mouse IgG antibodies (Zymed) were added to the plates. Color development was similarly carried out using the above method. Following a one-hour coloring reaction at room temperature, absorbance was measured at 405 nm and EC₅₀ values were computed using GraphPad Prism.

CHO-human Mpl cells and CHO-monkey Mpl cells were harvested, and suspended in FACS Buffer (1% FBS/PBS) to a final concentration of 1×10⁶ cells/mL. The suspensions were aliquoted into Multiscreen (Millipore) at 100 μl/well, and the culture supernatants were removed by centrifugation. Culture supernatants diluted to 5 μg/mL were added to the plates and incubated on ice for 30 min. The cells were washed once with FACS buffer, and incubated on ice for 30 min following the addition of an FITC-labeled anti-mouse IgG antibody (Beckman Coulter). After incubation, the mixture was centrifuged at 500 rpm for 1 min. The supernatants were removed, and then the cells were suspended in 400 μL of FACS buffer. The samples were analyzed by flow cytometry using EPICS ELITE ESP (Beckman Coulter). An analysis gate was set on the forward and side scatters of a histogram to include viable cell populations.

The mouse monoclonal antibodies VA130, VB16, and VB157 that bind to Mpl were obtained by evaluating their binding activity by ELISA using Mpl-Fc-immobilized plates, and flow cytometry using CHO-human Mpl and CHO-monkey Mpl.

1.7 Purification of Anti-Human Mpl Antibodies

Anti-human Mpl antibodies were purified from hybridoma culture supernatants as described below. After the culture supernatants were loaded onto HiTrap protein G HP columns (Amersham Biosciences) for adsorption, the antibodies were eluted with 0.1 M glycine-HCl (pH2.7) Buffer. After elution, the fractions were neutralized with 1 M Tris-HCl Buffer (pH9.0), and dialyzed against PBS to replace the buffer for one day.

### Example 2

### Preparation of Single-Chain Anti-Human Mpl Antibodies

Of the obtained anti-human Mpl antibodies, three types of antibodies showing high binding activity were selected and the single-chain antibody-expression systems for these antibodies were constructed using a genetic engineering technique. Examples for preparing single-chain antibodies from VA130 anti-human Mpl antibody are described below.

2.1 Cloning of the Anti-Human Mpl Antibody Variable Region

The variable region was amplified by RT-PCR using total RNA extracted from hybridomas producing anti-human Mpl antibodies. Total RNA was extracted from 1×10⁷ hybridoma cells using the RNeasy Plant Mini Kit (QIAGEN).

A 5′-terminal fragment of the gene was amplified from 1 μg of total RNA by the SMART RACE cDNA Amplification Kit (Clontech), using a synthetic oligonucleotide MHC-IgG1 (SEQ ID NO: 1) complementary to mouse IgG1 constant region or a synthetic oligonucleotide kappa (SEQ ID NO: 2) complementary to mouse K chain constant region. Reverse transcription was carried out at 42° C. for 1.5 hr.

The composition of the PCR reaction solution (50 μL in total) is shown below.

| | | |
|---|---|---|
| 10× Advantage 2 PCR Buffer (Clontech) | 5 | μL |
| 10× Universal Primer A Mix (Clontech) | 5 | μL |
| dNTPs (dATP, dGTP, dCTP, and dTTP) (Clontech) | 0.2 | mM |
| Advantage 2 Polymerase Mix (Clontech) | 1 | μL |
| Reverse transcription product | 2.5 | μL |
| Synthetic oligonucleotide, MHC-IgG1 or kappa | 10 | pmol |

The PCR reaction conditions were:

94° C. (initial temperature) for 30 sec;

five cycles of 94° C. for 5 sec and 72° C. for 3 min;

five cycles of 94° C. for 5 sec, 70° C. for 10 sec, and 72° C. for 3 min;

25 cycles of 94° C. for 5 sec, 68° C. for 10 sec, and 72° C. for 3 min; and final extension was at 72° C. for 7 min.

The PCR products were purified from agarose gel using the QIAquick Gel Extraction Kit (QIAGEN), and cloned into a pGEM-T Easy Vector (Promega). The nucleotide sequence was then determined using the ABI 3700 DNA Analyzer (Perkin Elmer). The nucleotide sequence of cloned VA130 H chain variable region (hereinafter abbreviated as “VA130-VH”) is shown in SEQ ID NO: 3 and the amino acid sequence encoded thereby is shown in SEQ ID NO: 4, and the nucleotide sequence of VA130 L chain variable region (hereinafter abbreviated as “VA130-VL”) is shown in SEQ ID NO: 5 and the amino acid sequence encoded thereby is shown in SEQ ID NO: 6.

2.2 Preparation of Expression Vectors for Anti-Human Mpl Diabodies

A gene encoding a VA130 single-chain Fv (hereinafter abbreviated as “VA130 diabody”) containing a five-amino acid linker sequence was constructed, by linking a nucleotide sequence encoding a (Gly₄Ser)₁ linker to the VA130-VH-encoding gene at its 3′ end and to the VA130-VL-encoding gene at its 5′ end; both genes had been amplified by PCR.

The VA130-VH forward primer, VA264-feco (SEQ ID NO: 7) was designed to contain an EcoRI site. The VA130-VH reverse primer, VA264-rL5 (SEQ ID NO: 8) was designed to hybridize to a DNA encoding the C terminus of VA130-VH, and to have a nucleotide sequence encoding the (Gly₄Ser)₁ linker and a nucleotide sequence hybridizing to the DNA encoding the N terminus of VA130-VL. The VA130-VL forward primer, VA264-fL5 (SEQ ID NO: 9) was designed to have a nucleotide sequence encoding the N terminus of VA130-VL, a nucleotide sequence encoding the (Gly₄Ser)₁ linker, and a nucleotide sequence encoding the C terminus of VA130-VH. The VA130-VL reverse primer, VA264-rflag (SEQ ID NO: 10) was designed to hybridize to a DNA encoding the C terminus of VA130-VL and to have a nucleotide sequence encoding a FLAG tag (Asp Tyr Lys Asp Asp Asp Asp Lys/SEQ ID NO: 11) and a NotI site.

In the first round of PCR, two PCR products: one containing VA130-VH and a linker sequence, and the other containing VA130-VL and the identical linker sequence, were synthesized by the procedure described below.

The composition of the PCR reaction solution (50 μL in total) is shown below.

| | | |
|---|---|---|
| 10× PCR Buffer (TaKaRa) | 5 | μL |
| dNTPs (dATP, dGTP, dCTP, and dTTP) (TaKaRa) | 0.4 | mM |
| DNA polymerase TaKaRa Ex Taq (TaKaRa) | 2.5 | units |
| pGEM-T Easy vector comprising VA130-VH or VA130-VL | 10 | ng |
| gene | | |
| Synthetic oligonucleotides, VA264-feco and VA264-rL5, | 10 | pmol |
| or VA264-fL5 and VA264-rflag | | |

The PCR reaction conditions were:
94° C. (initial temperature) for 30 sec;
five cycles of: 94° C. for 15 sec and 72° C. for 2 min;
five cycles of 94° C. for 15 sec and 70° C. for 2 min;
28 cycles of 94° C. for 15 sec and 68° C. for 2 min;
and final extension was at 72° C. for 5 min.

After the PCR products of about 400 bp were purified from agarose gel using the QIAquick Gel Extraction Kit (QIAGEN), the second-round PCR was carried out using aliquots of the respective PCR products according to the protocol described below.

The composition of the PCR reaction solution (50 μL in total) is shown below.

| | | |
|---|---|---|
| 10× PCR Buffer (TaKaRa) | 5 | μL |
| dNTPs (dATP, dGTP, dCTP, and dTTP) (TaKaRa) | 0.4 | mM |
| DNA polymerase TaKaRa Ex Taq (TaKaRa) | 2.5 | unit |
| First-round PCR products (two types) | 1 | μL |
| Synthetic oligonucleotides, VA264-feco and VA264-rflag | 10 | pmol |

The reaction conditions were:
94° C. (initial temperature) for 30 sec;
five cycles of 94° C. for 15 sec and 72° C. for 2 min;
five cycles of 94° C. for 15 sec and 70° C. for 2 min;
28 cycles of 94° C. for 15 sec and 68° C. for 2 min;
and final extension was at 72° C. for 5 min.

The PCR products of about 800 bp were purified from agarose gel using the QIAquick Gel Extraction Kit (QIAGEN), and then digested with EcoRI and NotI (both from TaKaRa). The resulting DNA fragments were purified using the QIAquick PCR Purification Kit (QIAGEN), and then cloned into pCXND3 to prepare pCXND3-VA130 db.

2.3 Preparation of Expression Vectors for Anti-Human Mpl Antibody sc(Fv)2

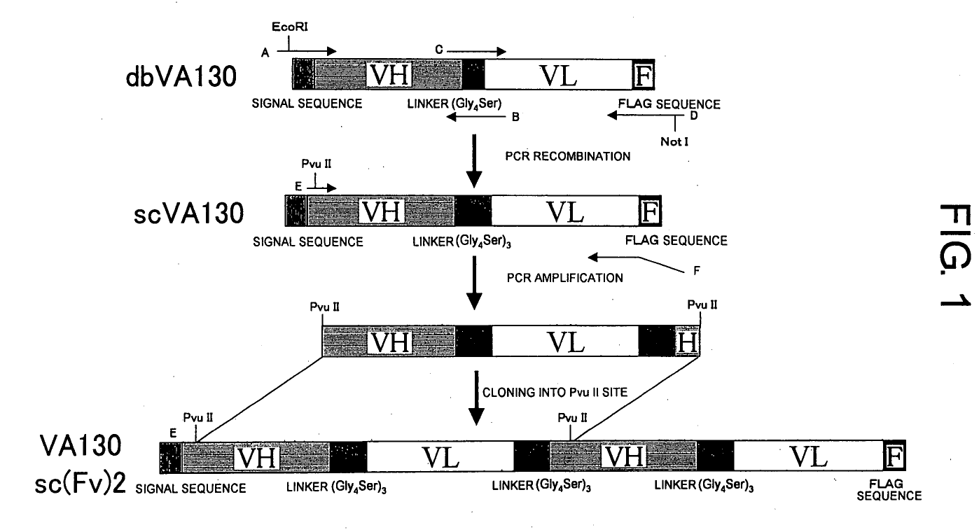

To prepare expression plasmids for the modified antibody [sc(Fv)2] comprising two units of H chain variable region and two units of L chain variable region derived from VA130, the above-described pCXND3-VA130 db was modified by PCR using the procedure shown below. The process for constructing the sc(Fv)2 gene is illustrated in FIG. 1.

First, PCR method was carried out to amplify (a) the VA130-VH-encoding gene in which a nucleotide sequence encoding a 15-amino acid linker (Gly₄Ser)₃ was added to its 3′ end; and (b) the VA130-VL-encoding gene containing the identical linker nucleotide sequence added to its 5′ end. The desired construct was prepared by linking these amplified genes. Two new primers were designed in this construction process. The VA130-VH reverse primer, sc-rL15 (primer B; SEQ ID NO: 12) was designed to hybridize to a DNA encoding the C terminus of VA130-VH, and to have a nucleotide sequence encoding the (Gly₄Ser)₃ linker, as well as a nucleotide sequence hybridizing to a DNA encoding the N terminus of VA130-VL. The VA130-VL forward primer, sc-fL15 (primer C; SEQ ID NO: 13) was designed to have a nucleotide sequence encoding the N terminus of VA130-VL, a nucleotide sequence encoding the (Gly₄Ser)₃ linker, and a nucleotide sequence encoding the C terminus of VA130-VH.

In the first-round PCR, two PCR products: one comprising VA130-VH and a linker sequence, and the other comprising VA130-VL and the identical linker sequence, were synthesized by the procedure described below.

The composition of the PCR reaction solution (50 μL in total) is shown below.

| | | |
|---|---|---|
| 10× PCR Buffer (TaKaRa) | 5 | μL |
| dNTPs (dATP, dGTP, dCTP, and dTTP) (TaKaRa) | 0.4 | mM |
| DNA polymerase TaKaRa Ex Taq (TaKaRa) | 2.5 | units |
| pCXND3-VA130 db | 10 | ng |
| Synthetic oligonucleotides, VA264-feco (primer A) and | 10 | pmol |
| sc-rL15, or sc-fL15 and VA264-rflag (primer D) | | |

The reaction conditions were:
94° C. (initial temperature) for 30 sec;
five cycles of 94° C. for 15 sec and 72° C. for 2 min;
five cycles of 94° C. for 15 sec and 70° C. for 2 min;
28 cycles of 94° C. for 15 sec and 68° C. for 2 min;
and final extension was at 72° C. for 5 min.

After the PCR products of about 400 bp were purified from agarose gel using the QIAquick Gel Extraction Kit (QIAGEN), the second-round PCR was carried out using aliquots of the respective PCR products according to the protocol described below.

The composition of the PCR reaction solution (50 μL in total) is shown below.

| | | |
|---|---|---|
| 10× PCR Buffer (TaKaRa) | 5 | μL |
| dNTPs (dATP, dGTP, dCTP, and dTTP) (TaKaRa) | 0.4 | mM |
| DNA polymerase TaKaRa Ex Taq (TaKaRa) | 2.5 | units |
| First-round PCR product (two types) | 1 | μL |
| Synthetic oligonucleotide, VA264-feco and VA264-rflag | 10 | pmol |

The reaction conditions were:
94° C. (initial temperature) for 30 sec;
five cycles of 94° C. for 15 sec and 72° C. for 2 min;
five cycles of 94° C. for 15 sec and 70° C. for 2 min;
28 cycles of 94° C. for 15 sec and 68° C. for 2 min;
and final extension was at 72° C. for 5 min.

The PCR products of about 800 bp were purified from agarose gel using the QIAquick Gel Extraction Kit (QIAGEN), and then digested with EcoRI and NotI (both from TaKaRa). The resulting DNA fragments were purified using the QIAquick PCR Purification Kit (QIAGEN), and then cloned into pBacPAK9 (Clontech) to construct pBacPAK9-scVA130.

A fragment to be inserted into the PvuII site of pBacPAK9-scVA130 was prepared. Specifically, the fragment has a PvuII recognition site at both ends and a nucleotide sequence, in which a gene encoding the VA130-VH N-terminus is linked, via a (Gly₄Ser)₃ linker-encoding nucleotide sequence, to a gene encoding the amino acid sequence of an N terminus-deleted VA130-VH linked to VA130-VL via the (Gly₄Ser)₃ linker. Two primers were newly designed to prepare the fragment by PCR. The forward primer for the fragment of interest, Fv2-f (primer E; SEQ ID NO: 14), was designed to have a PvuII site at its 5′ end and a VA130-VH 5′-end sequence. The reverse primer for the fragment of interest, Fv2-r (primer F; SEQ ID NO: 15), was designed to hybridize to a DNA encoding the C terminus of VA130-VL, and to have a PvuII site, a nucleotide sequence encoding the (Gly₄Ser)₃ linker, and a nucleotide sequence hybridizing to a DNA encoding the N terminus of VA130-VH. PCR was carried out using pBacPAK9-scVA130 as a template as described below.

The composition of the PCR reaction solution (50 μL in total) is shown below.

| | | |
|---|---|---|
| 10× PCR Buffer (TaKaRa) | 5 | μL |
| dNTPs (dATP, dGTP, dCTP, and dTTP) (TaKaRa) | 0.4 | mM |
| DNA polymerase TaKaRa Ex Taq (TaKaRa) | 2.5 | units |
| pBacPAK9-scVA130 | 10 | μg |
| Synthetic oligonucleotide, Fv2-f and Fv2-r | 10 | pmol |

The reaction conditions were:
94° C. (initial temperature) for 30 sec;
five cycles of 94° C. for 15 sec and 72° C. for 2 min;
five cycles of 94° C. for 15 sec and 70° C. for 2 min;
28 cycles of 94° C. for 15 sec and 68° C. for 2 min;
and final extension was at 72° C. for 5 min.

The PCR products of about 800 bp were purified from agarose gel using the QIAquick Gel Extraction Kit (QIAGEN), and then cloned into the pGEM-T Easy Vector (Promega). After sequencing, the plasmid was digested with PvuII (TaKaRa), and the fragment of interest was recovered. The recovered fragment was ligated to pBacPAK9-scVA130 pre-digested with PvuII (TaKaRa) to construct pBacPAK9-VA130 sc(Fv)2. After the resulting vector was digested with EcoRI and NotI (both from TaKaRa), the fragment of about 1,800 bp was purified from agarose gel using the QIAquick Gel Extraction Kit (QIAGEN). The fragment was then cloned into a pCXND3 expression vector to construct pCXND3-VA130 sc(Fv)2.

2.4 Expression of Single-Chain Anti-Human Mpl Antibody in Animal Cells

A cell line stably expressing the single-chain antibody was prepared from CHO-DG44 cells as described below. Gene transfer was achieved by electroporation using a Gene Pulser II (Bio-Rad). An expression vector (25 μg) and 0.75 mL of CHO-DG44 cells suspended in PBS (1×10⁷ cells/mL) were mixed. The resulting mixture was cooled on ice for 10 min, transferred into a cuvette, and pulsed at 1.5-kV and 25 μFD. After a ten-minute restoration period at room temperature, the electroporated cells were plated in CHO-S-SFMII medium (Invitrogen) containing 500 μg/mL Geneticin (Invitrogen). CHO cell lines expressing the single-chain antibody were established through selection. A cell line stably expressing VA130 sc(Fv)2 and its culture supernatants were obtained by this method.

The transient expression of the single-chain antibody was achieved using COS7 cells as described below. An expression vector (10 μg) and 0.75 mL of COS7 cells suspended in PBS (1×10⁷ cells/mL) were mixed. The resulting mixture was cooled on ice for 10 min, transferred into a cuvette, and then pulsed at 1.5-kV and 25 μFD. After a ten-minute restoration period at room temperature, the electroporated cells were plated in DMEM/10% FBS medium (Invitrogen). The cells were incubated overnight and then washed with PBS. CHO-S-SFMII medium was added and the cells were cultured for about three days. The culture supernatants for preparing the VA130 diabody were thus prepared.

2.5 Quantitation of Single-Chain Anti-Human Mpl Antibodies in Culture Supernatants

The culture supernatant concentration of the single-chain anti-human Mpl antibody transiently expressed in COS cells was determined using surface plasmon resonance. A sensor chip CM5 (Biacore) was placed in BIAcore 2000 (Biacore). ANTI-FLAG® M2 Monoclonal Antibody (Sigma-Aldrich) was immobilized onto the chip. An appropriate concentration of sample was injected over the chip surface at a flow rate of 5 mL/sec, and 50 mM diethylamine was used to dissociate the bound antibody. Changes in the mass during sample injection were recorded, and the sample concentration was calculated from the calibration curve prepared using the mass changes of a standard sample. db12E10 (see WO 02/33072 and WO 02/33073) was used as the diabody standard, and 12E10 sc(Fv)2 which has the same gene structure as that of sc(Fv)2 was used as the sc(Fv)2 standard.

2.6 Purification of Anti-Mpl Diabodies and Single-Chain Antibodies

The culture supernatants of VA130 diabody-expressing COS7 cells or CHO cells were loaded onto an Anti-Flag M2 Affinity Gel (Sigma-Aldrich) column equilibrated with a 50 mM Tris-HCl buffer (pH7.4) containing 150 mM NaCl and 0.05% Tween 20. The absorbed antibodies were eluted with 100 mM glycine-HCl (pH3.5). The fractions eluted were immediately neutralized with 1 M Tris-HCl (pH8.0), and loaded onto a HiLoad 26/60 Superdex 200 pg (Amersham Biosciences) column for gel filtration chromatography. PBS/0.01% Tween 20 was used in the gel filtration chromatography.

VA130 sc(Fv)2 was purified from the culture supernatants of VA130 sc(Fv)2-expressing COS7 cells or CHO cells under the same conditions used for purifying the diabodies. In each purification step, the presence of the diabody and sc(Fv)2 in the samples was confirmed by SDS-PAGE and Western blotting using an anti-Flag antibody (Sigma-Aldrich).

Specifically, obtained fractions corresponding to each peak were subjected to the electrophoresis according to the method described by Laemli, and then stained using Coomassie Brilliant Blue. As a result, single band was detected apparently at about 28 kDa for the diabody; while single band was detected apparently at about 58 kDa for sc(Fv)2.

2.7 Assessment of the Binding Activity of Anti-Human Mpl Single-Chain Antibody by Flow Cytometry

CHO-human Mpl, CHO-monkey Mpl, and CHO-mouse Mpl were harvested and suspended in FACS Buffer (1% FBS/PBS) at a cell density of 1×10⁶ cells/ml. The suspension was aliquoted into each well (100 μl/well) of Multiscreen-HV Filter Plates (Millipore) and the supernatant was removed after centrifugation. An appropriate concentration of diabody or sc(Fv)2 was added to each well and the plates were incubated on ice for 30 minutes. The cells were washed once with 200 μl of FACS buffer, then 10 μg/ml ANTI-FLAG M2 Monoclonal Antibody (SIGMA-ALDRICH) was added to the cells. The plates were incubated on ice for 30 minutes.

Then, the cells were washed once with 200 μl of FACS buffer, and 100-times-diluted FITC-labeled anti-mouse IgG antibody (Beckman Coulter) was added to the cells. The plates were incubated on ice for 30 minutes. Finally, after centrifugation and removal of the resulting supernatants, the cells were suspended in 400 μl of FACS Buffer, and fractionated by flow cytometry using EPICS ELITE ESP (Beckman Coulter). A gate was set for the population of viable cells on the forward scatter and side scatter histograms.

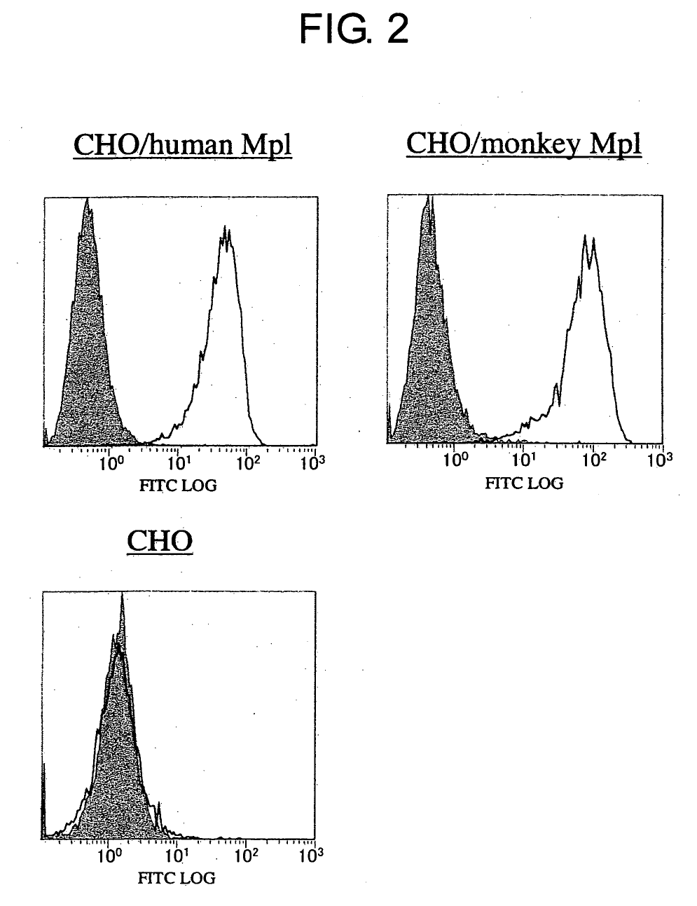

Various types of Mpl were expressed in CHO cells and binding activity to the CHO cells was assessed for purified VA130 sc(Fv)2. The results obtained are shown in FIG. 2. It was revealed that the antibody did not exhibit any binding activity to the host cell CHO, but bound specifically to CHO-human Mpl and CHO-monkey Mpl. This binding activity tendency was not different from that of VA130 IgG, and thus it was estimated that low-molecular-weight conversion did not alter the antibody binding site.

2.8 Assessment of the Binding Activity of Anti-Human Mpl Single-Chain Antibody by ELISA

The binding activity of anti-human Mpl single-chain antibody to hMPL-Fc was assessed using ELISA. After plates were coated with 0.5 μg/ml purified hMPL-Fc, blocking treatment was carried out using Diluent buffer. A purified product of VA130 was diluted to an appropriate concentration and added to the plates. Then, the plates were allowed to stand at room temperature for one hour. After washing with Rinse buffer, 1000-times-diluted ANTI-FLAG M2 Monoclonal Antibody (SIGMA-ALDRICH) was added. The plates were incubated at room temperature for one hour. Next, after washing with Rinse buffer, 1000-times-diluted Alkaline Phosphatase-labeled anti-mouse IgG antibody (Zymed) was added, and the plates were allowed to stand at room temperature for one hour. After washing with Rinse buffer, color development was achieved using SIGMA104 (SIGMA-ALDRICH) diluted to 1 mg/ml in substrate buffer. Color was developed for 15 minutes at room temperature and then absorbance was determined at 405 nm with Benchmark Plus.

2.9 Assessment of TPO-Like Agonist Activity for Single-Chain Anti-Human Mpl Antibodies

TPO-like agonist activity was assessed using BaF3-human Mpl that proliferate in a TPO-dependent manner. The cells were washed twice with RPMI 1640/1% FBS (fetal bovine serum) (Invitrogen), and then suspended in RPMI 1640/10% FBS to a concentration of 4×10⁵ cells/mL. Cell suspensions were aliquoted at 60 μL/well into a 96-well plate. Various concentrations of rhTPO (R&D) and COS7 culture supernatants or purified samples were prepared, and a 40 μL aliquot was added into each well. The plates were then incubated at 37° C. under 5% CO₂ for 24 hr. Immediately after a 10-μL aliquot of WST-8 reagent (Cell Count Reagent SF; Nacalai Tesque) was added into each well, absorbance was measured at 450 run (and at 655 nm as a control) using Benchmark Plus. After two hours of incubation, absorbance was again measured at 450 nm (and at 655 nm as a control). The WST-8 reagent changes colors at 450 nm in a color reaction that reflects the viable cell count. The TPO-like agonist activity was assessed using the change in absorbance during the two-hour incubation as an indicator. EC₅₀ values were computed using GraphPad Prism.

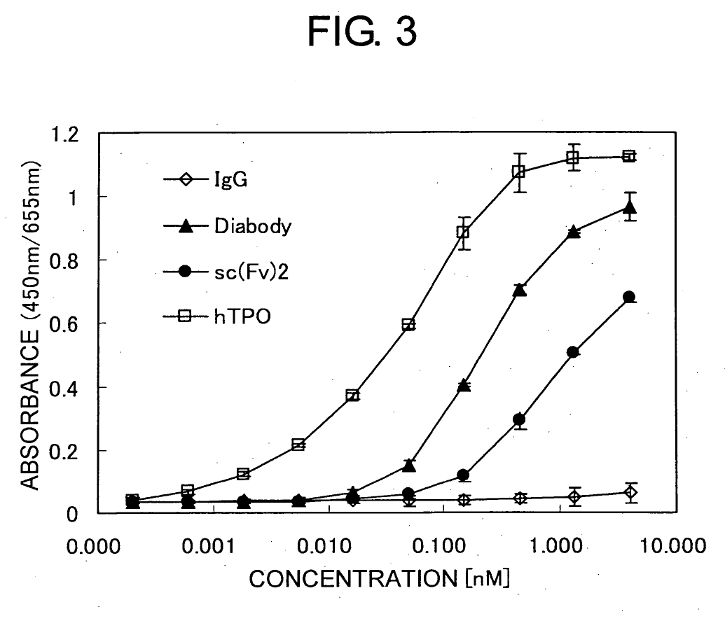

The TPO-like agonist activities of purified VA130 IgG, VA130 diabody, and VA130 sc(Fv)2 were assessed using BaF3-human Mpl. The results obtained are shown in FIG. 3.

VA130 IgG did not exhibit agonistic activity (BaF3-human Mpl EC₅₀: >100 nM). In contrast, the minibodies, VA130 diabody and VA130 sc(Fv)2, which were converted from VA130 IgG, exhibited agonistic activity (BaF3-human Mpl EC₅₀: 222 pM and 1023 pM, respectively). Table 1 shows an assessment of activity of VA130, VB16, and VB157. Like VA130, it was found that VB16 and VB157 IgGs exhibited strong binding activity but no agonistic activity; however, when converted to minibodies, both exhibited agonistic activity.

These results show that when producing agonist antibodies, screening using IgGs produced by hybridomas where agonistic activity is an indicator is not important, but that screening for agonist antibodies by converting receptor-binding antibodies to minibodies is important.

TABLE 1
Results of assessing the activities of VA130, VB16, and VB157

| | IgG | Diabody | | sc(Fv)2 | |
|---|---|---|---|---|---|
| | Binding | Binding | Agonistic | Binding | Agonistic |
| | activity | activity | activity | activity | activity |
| | ELISA | ELISA | Analysis | ELISA | Analysis |
| | using | using | using | using | using |
| Antibody | hMpl-Fc | hMpl-Fc | Baf-hMpl | hMpl-Fc | Baf-hMpl |
| VA130 | 0.38 nM | 0.11 nM | 222 pM | 0.27 nM | 1023 pM |
| VB16 | 0.15 nM | N.T. | 190 pM | N.T. | 95 pM |
| VB157 | 0.15 M | N.T. | 465 pM | N.T. | N.T. |

N.T.: Not tested

### INDUSTRIAL APPLICABILITY

In conventional screening methods, where agonistic activities of whole antibodies are determined prior to modification and antibodies without agonistic activity are excluded at this point, antibodies with no agonistic activity prior to modification are excluded at this point and only antibodies that have activity are modified. In such cases, it is impossible to discover antibodies that only have agonistic activity when converted to minibodies, and as a result minibodies derived from such antibodies are also undiscovered. The present inventors found that, even if whole antibodies have only weak or undetectable agonistic activity, the activity can be increased by conversion to minibodies.

The screening methods of the present invention do not select antibodies by using agonistic activity as an indicator prior to antibody modification, and therefore whole antibodies with only weak or undetectable agonistic activity are not excluded. Thus, antibodies with potentially higher activity, which are undetectable by previous methods, can be discovered using the methods of present invention.

1. A method of screening for an agonist antibody, which comprises the steps of:
(a) determining the binding activity of a test antibody and selecting an antibody with binding activity;
(b) modifying the antibody selected in step (a); and
(c) determining the agonistic activity of the modified antibody of step (b) and selecting an antibody with agonistic activity.

2. The screening method of claim 1, wherein the modified antibody is a minibody.

3. The screening method of claim 2, wherein the minibody is an sc(Fv)2.

4. The screening method of claim 1, wherein the agonistic activity is not determined prior to modifying the test antibody.

5. The screening method of claim 1, wherein the antibody is one against a protein expressed on a cell membrane.

6. An antibody obtained by the method of claim 1.

7. A method for producing an antibody with agonistic activity, which comprises the steps of:
(a) determining the binding activity of an antibody and selecting an antibody with binding activity;
(b) modifying the antibody selected in step (a);
(c) determining the agonistic activity of the modified antibody of step (b) and selecting an antibody with agonistic activity;
(d) introducing a host cell with a vector carrying a DNA that encodes the antibody selected in step (c); and
(e) culturing the host cell of step (d).

8. The production method of claim 7, wherein the modified antibody is a minibody.

9. The production method of claim 8, wherein the minibody is an sc(Fv)2.

10. The production method of claim 7, wherein the agonistic activity is not determined prior to antibody modification.

11. The production method of claim 7, wherein the antibody is one against a protein expressed on a cell membrane.

12. A method of screening for an agonist antibody, wherein the agonistic activity of a test antibody is not determined prior to step (a), and which comprises the steps of:
(a) modifying a test antibody; and
(b) determining the agonistic activity of the modified antibody of step (a) and selecting an antibody with agonistic activity.

13. The screening method of claim 12, wherein the modified antibody is a minibody.

14. The screening method of claim 13, wherein the minibody is an sc(Fv)2.


SEQUENCE LISTING (15 sequences)

SEQ ID NO: 1 - DNA, 21 bp, Artificial
  an artificially synthesized sequence
gggccagtggatagacagatg

SEQ ID NO: 2 - DNA, 23 bp, Artificial
  an artificially synthesized sequence
gctcactggatggtgggaagatg

SEQ ID NO: 3 - DNA, 411 bp, Mus musculus
  CDS (1)..(411)
atggaatggcctttgatctttctcttcctcctgtcaggaactgcaggtgtccactcccaggttcagctgcagcagtctggacctgagttggtgaagcctggggcctcagtgaagatttcctgcaaggcttctggctatgcattcagtagttcctggatgaactgggtgaagcagaggcctggaaagggtcttgagtggattggacggatttatcctggagatggagatactaactacaatgggaagttcaagggcaaggccacactgactgcagacaaatcctccagcacggcctacatacaactcagcagcctaacatctgaggactctgcggtctacttctgtgcaagagggtatgctgactactcctttgcttactggggccaagggactctggtcactgtctctgca

SEQ ID NO: 4 - protein, 137 aa, Mus musculus
MEWPLIFLFLLSGTAGVHSQVQLQQSGPELVKPGASVKISCKASGYAFSSSWMNWVKQRPGKGLEWIGRIYPGDGDTNYNGKFKGKATLTADKSSSTAYIQLSSLTSEDSAVYFCARGYADYSFAYWGQGTLVTVSA

SEQ ID NO: 5 - DNA, 396 bp, Mus musculus
  CDS (1)..(396)
atgaggtgcctagctgagttcctggggctgcttgtgctctggatccctggagccattggggatattgtgatgactcaggctgcaccctctgtacctgtcactcctggagagtcagtatccatctcctgcaggtctagtaagagtctcctgcatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgtatacgttcggatcggggaccaagctggaaataaaa

SEQ ID NO: 6 - protein, 132 aa, Mus musculus
MRCLAEFLGLLVLWIPGAIGDIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPYTFGSGTKLEIK

SEQ ID NO: 7 - DNA, 30 bp, Artificial
  an artificially synthesized primer sequence
tagaattccaccatggaatggcctttgatc

SEQ ID NO: 8 - DNA, 56 bp, Artificial
  an artificially synthesized primer sequence
agcctgagtcatcacaatatccgatccgcctccacctgcagagacagtgaccagag

SEQ ID NO: 9 - DNA, 56 bp, Artificial
  an artificially synthesized primer sequence
actctggtcactgtctctgcaggtggaggcggatcggatattgtgatgactcaggc

SEQ ID NO: 10 - DNA, 60 bp, Artificial
  an artificially synthesized primer sequence
attgcggccgcttatcacttatcgtcgtcatccttgtagtcttttatttccagcttggtc

SEQ ID NO: 11 - protein, 8 aa, Artificial
  an artificially synthesized FLAG tag sequence
DYKDDDDK

SEQ ID NO: 12 - DNA, 82 bp, Artificial
  an artificially synthesized primer sequence
tgagtcatcacaatatccgatccgccaccacccgaaccaccaccacccgaaccaccaccacctgcagagacagtgaccagag

SEQ ID NO: 13 - DNA, 82 bp, Artificial
  an artificially synthesized primer sequence
tggtcactgtctctgcaggtggtggtggttcgggtggtggtggttcgggtggtggcggatcggatattgtgatgactcaggc

SEQ ID NO: 14 - DNA, 25 bp, Artificial
  an artificially synthesized primer sequence
caggttcagctgcagcagtctggac

SEQ ID NO: 15 - DNA, 81 bp, Artificial
  an artificially synthesized primer sequence
gctgcagctgaacctgcgatccaccgcctcccgaaccaccaccacccgatccaccacctccttttatttccagcttggtcc